(19) United States
(12) Patent Application Publication
(10) Pub. No.: US 2011/0144544 A1
(43) Pub. Date: Jun. 16, 2011

(54) ULTRASOUND TRANSDUCER ASSEMBLY AND METHODS OF USING

(75) Inventors: Ying Fan, Niskayuna, NY (US); Warren Lee, Niskayuna, NY (US); Kenneth Wayne Rigby, Clifton Park, NY (US)

(73) Assignee: GENERAL ELECTRIC COMPANY, SCHENECTADY, NY (US)

(21) Appl. No.: 12/638,046

(22) Filed: Dec. 15, 2009

(51) Int. Cl. A61N 7/00

(52) U.S. Cl. 601/2

(57) ABSTRACT

An ultrasound transducer assembly for adjustably directing a beam of ultrasound energy to a region of interest is provided. The ultrasound transducer assembly comprises a therapy transducer, a beam enhancement component that adjusts one or more parameters of the ultrasound beam, wherein the beam enhancement component is in operative association with the therapy transducer, and a component controller operatively coupled to the beam enhancement component for controlling the beam enhancement component.

### BACKGROUND

The invention relates to ultrasound transducer assembly, and more particularly to ultrasound transducer assembly having therapy transducer with beam steering mechanisms and adjustable focal length and methods of using.

Ultrasound refers to acoustic waves having a frequency above the upper limit of the human audible range (i.e., above 20 kHz). Ultrasonic imaging is a widely used method for imaging and/or delivering therapy. In ultrasonic imaging or therapy, acoustic waves are generated by a transducer and directed towards a region of interest. In case of imaging applications, the resulting echo is detected by the transducer element(s) and analyzed by an imaging system to develop an image.

During ultrasound therapy, high voltage is applied to the ultrasound transducer to generate a correspondingly high acoustical output power. The typical ultrasound intensity (power per unit area) used in imaging is less than 0.1 watt per square centimeter. High intensity focused ultrasound (HIFU), which can have intensity above 1000 watts per square centimeter, can raise the tissue temperature at the region of the spatial focus to above 60 degrees Celsius in a few seconds producing tissue necrosis or lysis. Alternatively, the large negative pressure generated at the focus can destroy tissue through the mechanism of cavitation, that is, bubble generation, growth and collapse.

Certain treatments involving HIFU, such as non-invasive body contouring, require the use of a large HIFU transducer with the treatment occurring at the relatively small focus of the ultrasound energy. Typically, single-element hemispherical HIFU transducers are used in this application. Since the total treatment area is typically large with respect to the size of a single focal area, the transducer has to be physically moved by the clinician several hundred times to cover the entire treatment area. At each treatment location, ultrasound energy from the HIFU transducer is applied for a few seconds, allowing sufficient time either for a temperature rise in the case of thermal treatment or for bubble formation, growth and collapse in the case of cavitation-based treatment. The total treatment time depends upon the treatment time at each treatment location, the number of treatment locations, and the time required for the user to reposition the transducer at each treatment location. For non-invasive body contouring, it is desirable to minimize the total treatment time, since this improves patient comfort and lowers the treatment cost. Conversely, for a fixed total treatment time, it is desirable to maximize the total volume of tissue affected by the HIFU transducer focus.

The size of the focus of a high intensity focused ultrasound transducer is only a few mm to few cms. The size of the focus is controlled by ultrasound parameters such as transducer geometry and wavelength inside the tissue. Because of the small size of the focus many transducer movements are required to cover the entire region of interest since the region of interest is typically larger than the focus. Manual probe movement is typically required to adjust the probe position to each new location, perhaps including a trial and error approach by the user to reach and verify the appropriate location, thereby causing discomfort to the patient and increasing the time for imaging or therapy.

One potential solution to the problems described above is a therapy transducer phased array, which allows electronic steering of the focus of the ultrasound beam. The rapid electronic steering such an array allows would permit multiple focal spots to be treated from a single transducer location, avoiding the time-consuming step of repositioning the transducer.

However, system and transducer complexity is still one of the major disadvantages of electronic therapeutic arrays. A therapeutic transducer requires a large surface area to generate a high acoustic power. Preferably, the f-number (focal depth divided by aperture size) is kept constant within the range from 0.8 to 2.5. On the other hand, to steer the ultrasound beam over a wide range and to focus the beam, the ultrasound phased array must have array elements with dimensions on the order of a wavelength of the ultrasound wave. Consequently, a therapeutic array transducer would typically require hundreds or even thousands of elements.

Usually, each element has a dedicated electronic driving circuit in a control system for the array. To drive a phased array like that discussed, the control system would need to include hundreds of sets of driving circuits, one for each element. The array and the control system are connected through a large cable that includes these hundreds of smaller cables inside it. Each smaller cable should have conductors of a sufficiently large cross-sectional area to carry a relatively large current to the therapeutic array element. The thick cable required to meet this need makes the device difficult to handle. Considering all these constraints, it will be evident that the complexity of such a therapeutic phased array, including the cable and the control system coupled to it, can easily become impractical to implement, and the cost also increases. For these reasons, the therapeutic phased array has not been widely accepted.

Therefore, it would be desirable to provide a simple and accurate solution for adjusting ultrasound beam steering and focus spot scanning of the ultrasound to use for therapy purposes.

### BRIEF DESCRIPTION

In one embodiment, an ultrasound transducer assembly for adjustably directing a beam of ultrasound energy to a region of interest is provided. The ultrasound transducer assembly comprises a therapy transducer, a beam enhancement component that adjusts one or more parameters of the ultrasound beam, wherein the beam enhancement component is in operative association with the therapy transducer, and a component controller operatively coupled to the beam enhancement component for controlling the beam enhancement component.

In another embodiment, an ultrasound transducer system is provided. The system comprises a probe housing, an ultrasound transducer assembly at least partially disposed in the probe housing. The probe housing comprises a therapy transducer, a beam enhancement component that adjusts one or more parameters of the ultrasound beam, wherein the beam enhancement component is in operative association with the therapy transducer, and a component controller operatively coupled to the beam enhancement component for controlling the beam enhancement component. The system further includes a therapy module that provides one or more therapy parameters to the ultrasound transducer assembly.

In yet another embodiment, a method for non-invasively providing therapy to a region of interest using an ultrasound transducer associated with a probe housing is provided. The method comprises generating an ultrasound beam, directing at least a portion of the beam to a portion of the region of interest, and adjustably directing the same portion, or a different portion, of the ultrasound beam to another portion of the region of interest without moving the probe housing.

### DRAWINGS

FIG. 1 is a block diagram of an example of an example of an ultrasound therapeutic and imaging system;

FIG. 2 is a schematic diagram of an example of ultrasound system for non-invasively providing ultrasound based therapy to a patient using the ultrasound transducer assembly of the present invention;

FIGS. 3-16 are cross-sectional views of alternate embodiments of ultrasound transducer assemblies of the present invention; and

FIG. 17 is a flow chart for an example of a method of the invention for non-invasively providing therapy to a region of interest using the ultrasound transducer assembly of the present invention.

These and other features, aspects, and advantages of the present invention will become better understood when the following detailed description is read with reference to the accompanying drawings in which like characters represent like parts throughout the drawings, wherein:

### DETAILED DESCRIPTION

Embodiments of the invention provide a simplified and inexpensive solution for varying the shape and position of the focal region of an ultrasonic beam. In certain embodiments, the ultrasound system incorporates a scheme for focusing and steering ultrasonic energy emitted by the ultrasound transducer to a region of interest. In certain embodiments, the ultrasound system of the invention comprises an ultrasound transducer assembly that may be used to deliver therapy to a region of interest. In certain embodiments, the ultrasound transducer assembly comprises a therapy transducer, a beam enhancement component that adjusts a parameter of the ultrasound beam, wherein the beam enhancement component is in operative association with the therapy transducer, and a component controller operatively coupled to the beam enhancement component for controlling the movement of the beam enhancement component. The ultrasound transducer assembly may be at least partly disposed in a probe housing. For example, the therapy transducer and the beam enhancement component may be disposed in the probe housing, and the component controller may be disposed outside the probe housing.

In certain embodiments, it may be desirable to shorten the therapy time by overcoming the need for manual physical movement of the probe for every desirable shift in position for the focus of the ultrasound beam. The beam enhancement component and the associated controller enable the ultrasound transducer assembly to generate an ultrasound beam having an adjustable shape, or size, or direction, or location, or combinations thereof. The ultrasound transducer assembly can cover a plurality of regions of interest, while the probe housing remains disposed at the same physical location on the subject (such as a patient). For example, the acoustic beam from the transducer can be steered to cover a plurality of regions of interest or a single large region of interest. In one embodiment, the ultrasound transducer assembly may have more than one focal depth. For example, the ultrasound transducer assembly may have adjustable focal depth. The transducer may have a single element or an array of elements. In certain embodiments, the transducer elements share one transmitting system (a single channel), thereby simplifying the electrical system design and also reducing the manufacturing cost for the ultrasound transducer assembly.

To more clearly and concisely describe the subject matter of the claimed invention, the following definitions are provided for specific terms, which are used in the following description and the appended claims. Throughout the specification, exemplification of specific terms should be considered as non-limiting examples.

As used herein, “cavitating” refers to treating tissue (such as fat tissue) primarily with cavitational mechanism. However, cavitating may also have thermal effects on the tissue. The tissue may be destroyed either by cavitation or thermal effect. HIFU parameters may be adjusted such that the majority of the damage to tissue is cavitational in nature, but there may be thermal effects or damages to the tissue.

As used herein, “thermally treating” refers to treating tissue primarily with a mechanism that is thermal in nature. However, thermally treating may have cavitational effects.

As used herein, “adipose tissue” means subcutaneous, visceral or other tissues made primarily of fat cells. Adipose tissues may also comprise connective tissues, blood vessels and other structures. Adipose tissue may be white adipose tissue or brown adipose tissue.

As used herein, “connective tissues” are found either in the adipose tissue or in the skin surrounding the adipose tissue.

As used herein, the term “beam enhancement component” refers to a component that adjusts a parameter (such as shape, size, location, or direction) of the ultrasound beam. In one embodiment, the beam enhancement component may move the transducer or transducer elements along or about one or more of x, y or z directions to adjust the parameter of the ultrasound beam. For example, the beam enhancement component may rotate the transducer about the z direction, or enable a lateral displacement of the transducer along x or y direction. In another embodiment, the beam enhancement component may move an optics (such as a lens) operatively coupled to the transducer along or about an x, y, or z-axis to adjust the parameter of the ultrasound beam. “Move” may mean translational movement, or rotational movement, or both.

As used herein, “patient” or “subject” means a person or living being receiving therapy using the ultrasound transducer assembly. The terms “patient” and “subject” may be used interchangeably throughout the application.

As used herein, “region of interest” means one or more sites associated with the patient targeted for receiving the therapy. The region of interest may or may not be imaged along with the therapy treatments. The region of interest may include, but is not limited to, an inner (deeper) treatment region, a subcutaneous region of interest and/or any other region of interest in between the inner treatment region, and/or a subcutaneous region within a patient.

As used herein, “therapy transducer” refers to an ultrasound transducer that generates ultrasound energy for therapeutic purposes to be delivered to a region of interest.

As used herein, “user” means one or more persons (e.g. skilled technician or physician) operating at least part of the system to provide therapy to the patient.

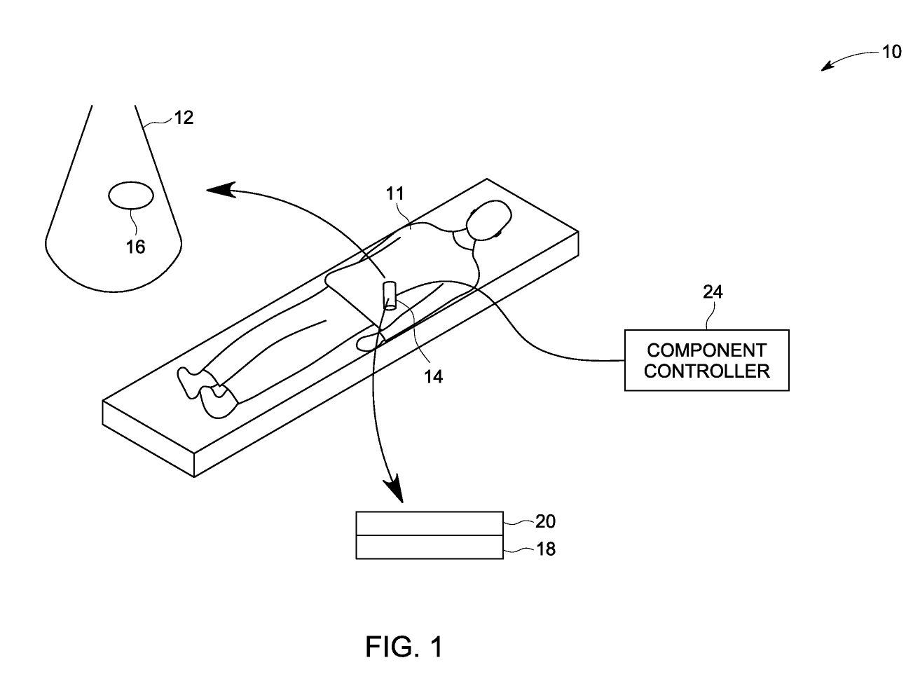

FIG. 1 is a block diagram of an example of a system 10 for providing therapy. The figures are for illustrative purposes and are not drawn to scale. The system 10 may be configured to non-invasively deliver therapy and/or to image a region of interest 12 via a probe housing 14. The region of interest 12 may be a one-dimensional, two-dimensional or a three dimensional region. The region of interest 12, having diseased tissue or adipose tissue, for example, is located inside a patient 11. In one example, providing the therapy may include destroying the diseased tissues or adipose tissues 16 in the region of interest 12 by heating or by inducing cavitation.

The probe housing 14 comprises an ultrasound transducer assembly comprising a therapy transducer 18 and a beam enhancement component 20 that is operatively coupled to the transducer 18. In one embodiment, both the transducer 18 and the beam enhancement component 20 may be disposed in a coupling fluid through which the acoustic waves can travel. In another embodiment, the transducer 18 may be disposed in the coupling fluid, but the beam enhancement component 20 may not be in the coupling fluid. For instance, a motor could be sealed from the coupling fluid, but a drive shaft could extend through the seal and attach to the transducer 18. The beam enhancement component 20 is controlled using a component controller 24. Specifically, the controller 24 is operatively coupled to the beam enhancement component 20 to control the parameters of the ultrasound beam, such as a shape, size, or direction of the beam. For example, the controller 24 may move the beam enhancement component 20 in a lateral direction, or rotate or oscillate the beam enhancement component 20 about an axis. In certain embodiments, the transducer may undergo circular or elliptical motion along with the beam enhancement component 20. Moreover, the transducer may exhibit lateral motion to affect the dynamic focusing and beam steering effects, for example.

The component controller 24 may also direct the beam enhancement component 20 to be switched on and off. When the beam enhancement component 20 is switched on, it may be directed by the controller to make a specific movement, such as a lateral movement along one of the axis, or a rotation along an axis, or both. In one embodiment, the transducer 18 may continue to provide acoustic radiation to a region of interest in the absence of any signals from the controller 24 to the beam enhancement component 20.

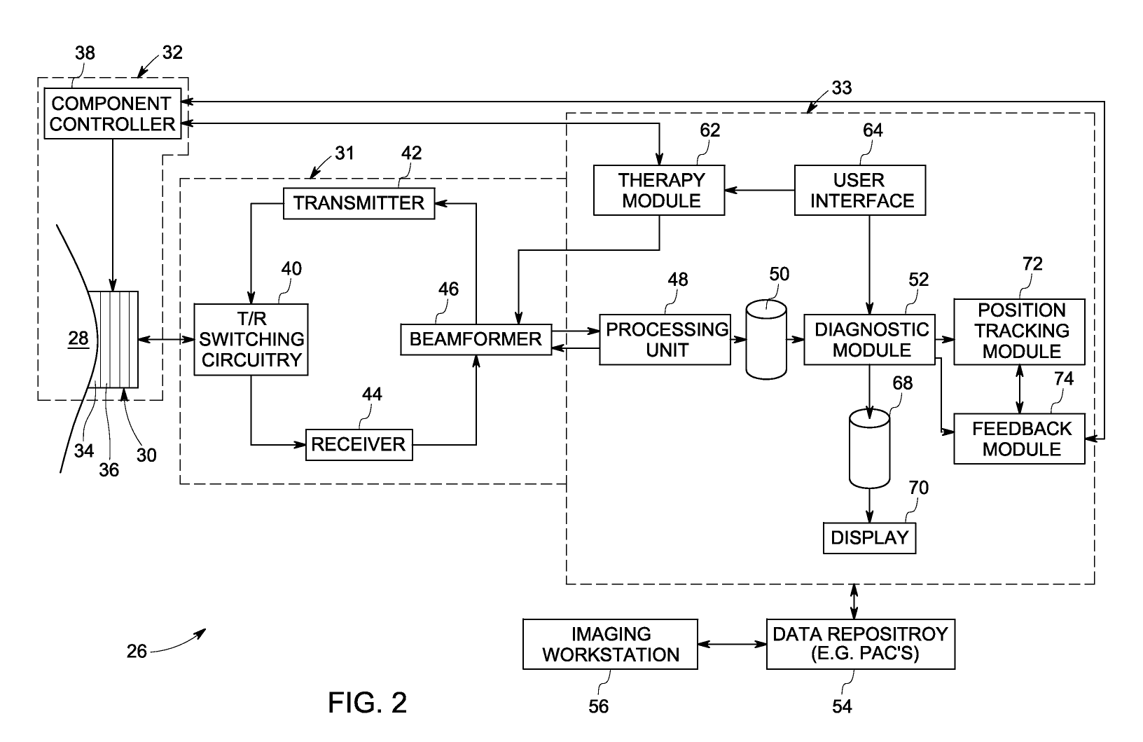

FIG. 2 illustrates an example of an ultrasound system 26 for delivering therapy and optionally imaging a region of interest 28. In this embodiment, system 26 enables real time dynamic focusing and beam steering of an ultrasonic beam. The ultrasound system 26 comprises an acquisition subsystem 31 and a processing subsystem 33. The system 26 also comprises an ultrasound transducer assembly 32 that partly forms the acquisition subsystem 31. In this example, the ultrasound transducer assembly 32 is partially disposed in a probe housing 30.

The ultrasound transducer assembly 32 comprises a therapy transducer 34, a beam enhancement component 36 and a component controller 38 for controlling the movements of the beam enhancement component 36.

The acquisition subsystem 31 comprises transmit/receive switching circuitry 40, a transmitter 42, and (optionally) a receiver 44. For therapy transducer 34, the receiver 44 may not be required. However, if an integrated imaging transducer is used in conjunction with the therapy transducer 34, the receiver 44 may be used to acquire back-scattered acoustic energy from the region of interest for imaging purposes. The imaging transducer may receive the backscattered waves at different times, depending on the distance into the tissue they return from and the angle with respect to the surface of the imaging transducer at which they return. The transducer elements convert the ultrasound energy from the backscattered waves into electrical signals. The electrical signals are then routed through the T/R switching circuitry 40 to the receiver 44. The receiver 44 amplifies and digitizes the received signals and provides other functions such as gain compensation. The digitized received signals, corresponding to the backscattered waves, are received, in this example, by each transducer element at various times, and preserve the amplitude and phase information of the backscattered waves.

In this example of the acquisition subsystem 31, the probe housing 30 is in physical contact with the patient 28. The transducer 34 is coupled to the transmit/receive (T/R) switching circuitry 40. Also, the T/R switching circuitry 40 is in operative association with an output of the transmitter 42 and an input of the receiver 44.

In one embodiment, the ultrasound transducer assembly 32 may be partially disposed in the probe housing 30. For example, the therapy transducer 34 and the beam enhancement component 36 may be disposed in the probe housing, and the component controller 38 may be disposed outside the probe housing 30. In these embodiments, the beam enhancement component 36 enables the therapy transducer 34 to move a determined lateral distance, or make determined angular motion in response to a signal generated by the component controller. A position-sensing device is operatively coupled to the transducer or the probe housing 30 to accurately determine the transducer position.

In some embodiments, the beam enhancement component 36 may be an actuator assembly. The actuator assembly may comprise a mechanical drive member, such as a motor, in operative association with the therapy transducer 34. In one embodiment, the transducer is coupled to a shaft of the mechanical driver member; such as drive motor, and disposed in the probe housing 30, thereby enabling the mechanical drive member to directly rotate the therapy transducer 34. In one embodiment, the therapy transducer 34 and the beam enhancement component 36 are disposed in a coupling fluid. In an alternate embodiment, the beam enhancement component may be disposed outside the coupling fluid.

In other embodiments, the control of the focal position may be accomplished using a beam enhancement component. The beam enhancement component may include a focusing element, such as a lens, acoustically coupled to the therapy transducer 34, where the focusing element adjusts the beam parameter. For example, an acoustic asymmetric lens having a non-uniform thickness and contoured surface is disposed in the path of the ultrasonic beam traveling from the therapy transducer 34 to the region of interest. The lens may be made to undergo motion in a plane substantially normal to the direction of the therapy beam thereby changing the lens characteristics intercepting the therapy beam in a predetermined manner for changing the path and focal zone length of the therapy beam. The lens may be rotated or oscillated about an axis to adjust the beam parameters. Alternatively or in addition, the lens may be laterally moved about an axis. For example, the lens may be moved about the z-axis to change the focal depth, and size of the beam.

The lens may move about an axis to adjust the beam parameter. In one embodiment, a matching layer or a compliant member may be disposed between the beam enhancement component 36 and the transducer 34. In one example, a compliant layer may be disposed between the mechanical drive member and the transducer 34 to prevent slippage of the mechanical drive member and the transducer 34. In another example, the compliant member may be disposed between the therapy transducer 34 and the acoustic lens to minimize reflection of the energy, for example, as energy passes from the therapy transducer 34 towards the lens. In some embodiments, the beam enhancement component is required to be compact enough to be disposed in the probe housing 30. In some embodiments, the component controller 38 may be a microprocessor-based controller.

In certain embodiments, the transducer 34 may comprise a single element or a plurality of transducer elements (not shown) arranged in a spaced relationship to form a transducer array, such as a one or two-dimensional transducer array, or annular array, for example. The transducer 34 may employ a single channel or multiple channels for transmission. Additionally, the acquisition sub-system 31 may comprise an interconnect structure (not shown) configured to facilitate operatively coupling the transducer array to an external device (not shown), such as, but not limited to, a cable assembly or associated electronics. In the illustrated embodiment, the interconnect structure may be configured to couple the transducer array to the T/R switching circuitry 40.

The imaging signals are back-scattered from physiological structures in the body, for example, adipose tissue, muscular tissue, blood cells, veins or objects within the body (e.g., a catheter or needle) to produce echoes that return to the imaging transducer. The echoes are received by the receiver 44 for imaging purposes. The received echoes are provided to a beamformer 46 that performs beamforming and outputs a signal. The signal from the beamformer 46 is then provided to a processor unit 48 that processes the signals. The processor unit 48 may include a complex demodulator (not shown) that demodulates the signals from the beamformer 46 to create pairs of I and Q demodulated data values representative of echo signals and corresponding to sample volumes along the scan line. Demodulation is accomplished by comparing the phase and amplitude of the received beam signals to a reference frequency. The I and Q demodulated data values preserve the phase and amplitude information of the received signals. The signal data may be transmitted to a memory 50 for storage (e.g., temporary or permanent storage). The output of the beamformer 46 may also be passed to the diagnostic module 52.

Optionally, the processing subsystem 33 may be coupled to a data repository 54 configured to receive ultrasound image data. The data repository 54 interacts with an imaging workstation 56. The processing subsystem 33 or the processor unit 48 alone may be coupled to a remote connectivity subsystem 58 including a web server 60 and a remote connectivity interface 62. The components may be dedicated hardware elements such as circuit boards with digital signal processors or may be software running on a general-purpose computer or processor such as a commercial, off-the-shelf personal computer (PC). The various components may be combined or separated according to various embodiments of the invention. The ultrasound system 26 is an example, and the systems and methods of the invention are not limited by this specific system configuration.

The ultrasound system 26 transmits ultrasound energy into the patient 28 to treat and/or image the region of interest, and receives and processes backscattered ultrasound signals from the patient 28 to create and display an image. To generate a transmitted beam of ultrasound energy, the processor unit 48 sends command data to the beamformer 42 to generate transmit parameters to create a beam of a desired shape originating from a certain point at the surface of the transducer 34 at a desired steering angle. The component controller 38 communicates with the beam enhancement component 36 to control the movement of the beam enhancement component 36, and consequently the movement of the transducer 34 to desirably steer and focus the beam. The transmission parameters are delivered from the beamformer 46 to the transmitter 42. The transmitter 42 uses the transmission parameters to properly encode the signals to be sent to the transducer 34 through the T/R switching circuitry 40. The signals are set at certain levels and phases with respect to each other and are transmitted to transducer element(s) of the transducer 34. The transmitted signals excite the transducer element(s) to emit ultrasound waves with the same phase and level relationships. As a result, a transmitted beam of ultrasound energy is directed at therapy site of the patient 28 along a scan line when the transducer 34 is acoustically coupled to the therapy site of the patient 28 by using, for example, ultrasound gel. The process is referred to as electronic scanning.

The digitized signals are sent to the beamformer 46. The processor unit 48 sends command data to beamformer 46. The beamformer 46 uses the command data to form a receive beam originating from a point on the surface of the transducer 34 at a steering angle typically corresponding to the point and steering angle of the previous ultrasound beam transmitted along a scan line. In this example, the beamformer 46 operates on the appropriate received signals, based on time delay and focusing parameters, according to the instructions of the command data from the processor unit 48, to create received beam signals corresponding to sample volumes along a scan line at the therapy site of patient 28. The phase, amplitude, and timing information of the received signals from the various transducer elements is used to create the received beam signals.

The demodulated data is transferred to the imaging mode processor 48. The imaging mode processor 48 uses parameter estimation techniques to generate imaging parameter values from the demodulated data in scan sequence format. The imaging parameters may include, but are not limited to, parameters corresponding to various possible imaging modes such as B-mode, color velocity mode, spectral Doppler mode, and tissue velocity imaging mode. The imaging parameter values are passed to a scan converter. The scan converter processes the parameter data by performing a translation from scan sequence format to display format. The translation comprises performing interpolation operations on the parameter data to create display pixel data in the display format. The scan converted pixel data is sent to the display processor to perform any final spatial or temporal filtering of the scan converted pixel data, to apply grayscale or color to the scan converted pixel data, and to convert the digital pixel data to analog data for display on the monitor 70.

The therapy or imaging transducers may include one or more transducer elements, one or more matching layers, and focusing components, such as lens. The transducer elements may be arranged in a spaced relationship, such as, but not limited to, an array of transducer elements disposed on a layer, where each of the transducer elements may include a transducer front face and a transducer rear face. The transducer elements may comprise, but are not limited to, a piezoelectrically active material, such as lead zirconate titanate (PZT), lithium niobate, lead titanate, barium titanate, and/or lead metaniobate, or combinations thereof. The piezoelectrically active component of the transducer element may also, or alternatively, comprise one or more of a piezoelectric ceramic, a piezoelectric crystal, piezoelectric plastic, and/or piezoelectric composite materials. In addition to, or instead of, a piezoelectrically active material, transducer 34 may comprise any other materials configured for generating radiation and/or acoustical energy such as capacitively coupled transducers or other acoustic sources. The transducer 34 may also comprise one or more matching and/or backing layers configured along with the transduction element such as coupled to the piezoelectrically active material. The therapy transducer 34 may also include one or more matching layers disposed adjacent to the front face of the array of transducer elements, where each of the matching layers may include a matching layer front face and a matching layer rear face. The matching layers facilitate acoustic impedance matching of the differential that may exist between the high impedance transducer elements and a low impedance patient 28.

The therapy transducer 34 may operate in a range of frequencies depending on the therapy desired. For example, the therapy transducer may operate in a frequency range from about 200 KHz to about 2 MHz for cavitating the unwanted tissues (such as diseased tissues or adipose tissues).

The imaging transducer may image the region of interest before applying the therapy, or after applying the therapy, or while applying the therapy. In one example, the therapy transducer generates one or more ultrasound frequencies for cavitating and/or thermally treating the region of interest, and the imaging transducer may receive one or more frequencies for imaging the region of interest. The therapy transducer and/or the imaging transducer may be configured to operate in a plurality of regions of interest while maintaining the same physical location of the probe housing on the subject.

A therapy module 62 is used to control the delivery of therapy to the treatment locations based on one or more therapy parameters or therapy commands. In one example, the transducer 34 may image the region of interest to determine therapy parameters pertaining to the adipose tissue and the non-adipose tissues. The therapy parameters may be generated by the therapy module 62 and transmitted to the acquisition sub-system 31. The component controller 38 may receive therapy parameters from the therapy module 62 and control the movement of the beam enhancement component 36 accordingly. The therapy module 62 may be connected to a user interface 64, such as a mouse, keyboard, and controls operation of the probe housing 30. For example, the user interface 64 is coupled to the therapy module 62 to allow a user to interface with the ultrasound system 26 based on the data displayed on the display 70, such as a monitor.

The therapy module 62 and/or the component controller 38 may be configured to receive inputs from a user using the user interface 64. For example, the user may provide instructions on whether to image the region of interest, or provide therapy to the region of interest. Further, the user can specify whether to cavitate or provide thermal treatment for the region of interest. The therapy module 62 and/or the component controller 38 may receive imaging and/or therapy commands from the user through a user interface 64 for applying therapy to the region of interest. The delivery of therapy may be based upon therapy commands provided by the user or the therapy parameters provided by the therapy module 62. The user interface 64 may be a touch screen, allowing the operator or user to select options by touching displayed graphics, icons, and the like.

A therapy command may comprise any factor or value that may be determined by the system 26 or any input that may be entered by the user that affects the therapy applied to the region of interest. In some embodiments, the system 26 may automatically differentiate the adipose tissue and the non-adipose tissue (such as connective tissue). The system 26 may also automatically display to a viewer (such as the user 44 or the patient) a boundary between the adipose tissue and between the adipose tissue and the non-adipose tissue by overlaying the image with a graphical representation that indicates the boundary. Furthermore, the system 26 may automatically display to a viewer the region of interest within the image where therapy may be applied (or is recommended by the system 26 to be applied). In addition, the user may be able to modify the treatment space that was automatically displayed by the system 26 through user inputs.

A therapy command may include a transducer parameter that relates to the configuration or operation of the transducer elements (not shown) or probe 30. Examples of the therapy command may include parameters of the ultrasound transducer 34 or time period for applying the therapy. The terms “therapy commands” and “therapy parameters” may be used interchangeably throughout the application, and refer to the settings of the system or the factors regarding the patient that are taken into account for delivering the therapy. A variety of geometries may be used and the probe 30 may be provided as part of, for example, different types of ultrasound probes. For example, therapy command may include instructions to the system 26 to deliver low energy pulses during imaging and high-energy pulses during therapy.

Examples of a transducer parameter include, but are not limited to, a focal region depth, a focal region size, an ablation time for each point within the region of interest that receives therapy, an energy level of the therapy signals, and a rate of focal region movement within the ROI during the therapy session. The transducer parameters may also include a frequency or intensity of the therapy ultrasound signals, power, peak rare factional pressure, pulse repetition frequency and length, duty cycle, depth of field, waveform used, speed of beam movement, density of beam, therapy pulse sequence, and imaging pulse sequence parameters. Also, therapy commands may include anatomical parameters, such as the location, shape, thickness, and orientation of adipose tissue and non-adipose tissues. An anatomical parameter may also include a density of the adipose tissue and the non-adipose tissues. Furthermore, therapy parameters include the type of probe 30 used during the therapy session. The age, gender, weight, ethnicity, genetics, or medical history of the patient may also be therapy commands. After therapy has been applied to a region of interest, the system 26 or the operator/user may adjust the therapy parameters before applying therapy to the same region of interest 28 again, or to a new region of interest.

The therapy module 62 or the diagnostic module 52 may be implemented by utilizing any combination of dedicated hardware boards, DSPs, processors, etc. Alternatively, the component controller 38 or the processing unit 48 may be implemented utilizing an off-the-shelf PC with a single processor or multiple processors, with the functional operations distributed between the processors. As a further option, the modules 52 and 62 may comprise a hybrid configuration in which certain modular functions are performed utilizing dedicated hardware, while the remaining modular functions are performed utilizing an off-the-shelf PC and the like.

The data may be processed by the processor unit 48 by employing one or more of a color-flow module, an acoustic radiation force imaging (ARFI) module, a B-mode module, a spectral Doppler module, an acoustic streaming module, a tissue Doppler module, a C-scan module, and an elastography module. Other modules may be included, such as an M-mode module, power Doppler module, harmonic tissue strain imaging, among others. However, embodiments of the systems and methods are not limited to processing IQ data pairs. For example, processing may be carried out with RF data and/or using other methods. Furthermore, data may be processed through multiple modules.

Each of the modules 52 and 62 are configured to process the IQ data pairs in a corresponding manner to generate color-flow data, ARFI data, B-mode data, spectral Doppler data, acoustic streaming data, tissue Doppler data, C-scan data, elastography data, among others, all of which may be stored in a memory 50 or 68 temporarily before subsequent processing. As an example, it may be desired to view different ultrasound images relating to a therapy session in real-time on the display 70.

The processor unit 48 processes the acquired ultrasound information (e.g., RF signal data or IQ data pairs) and prepares frames of ultrasound information for display on the display 70. The display 70 comprises one or more monitors that display patient information, such as including diagnostic and therapeutic ultrasound images, to the user for review, diagnosis, analysis, and treatment. The display 70 may automatically display, for example, 2D, 3D, and/or 4D ultrasound data stored in the memory 68 or currently being acquired, this stored data may also be displayed with a graphical representation (e.g., an outline of a treatment space or a marker within the treatment space).

The processing unit 48 is adapted to perform one or more processing operations according to a plurality of selectable ultrasound modalities on the acquired ultrasound information. Acquired ultrasound information may be processed in real-time during a scanning or therapy session as the echo signals are received. Additionally or alternatively, the ultrasound information may be stored temporarily in the memory 50 during a scanning session and processed in less than real-time in a live or off-line operation. The image memory 68 may be used to store processed frames of acquired ultrasound information that is not scheduled to be displayed immediately, for example. The image memory 68 may comprise any known data storage medium, for example, temporary or permanent storage mediums or removable storage mediums.

Optionally, the system 26 may include a position tracking module 72 that tracks a position of the probe 30 or the focus spot of the ultrasonic beam and communicates the position to the diagnostic module 52, for example. A position of the probe 30 may be tracked relative to a reference point on or near the patient, a marker, and the like. In one example, the position of the probe 30 may be used to indicate, to the user, regions of the patient that have already been treated, are being treated, or have yet to be treated.

After or while providing therapy to an area within the region of interest 28, the user may determine, whether the therapy is complete for the region of interest 28 and if the focus spot of the ultrasonic beam should be moved to another point within the patient. Automatic determination of whether the treatment space has been sufficiently treated or completed may be determined by, for example, elasto-graphic methods. Alternatively, the user or a feedback module 74 may determine whether the therapy is complete. The feedback module 74 may also be linked to the position tracking module to determine whether the focus spot is at the desired position.

The feedback module 74 may be coupled to the diagnostic module 52, or the component controller 38, or the therapy module 62. In addition, although not illustrated, the feedback module 74 may also be coupled to one or more of the display 70, memory 50, image memory 68, and the user interface 64. The feedback module 74 may compare the actual output of the system with the desired output. The actual output refers to the result of the therapy delivered to the region of interest. The actual output may be provided as displayed images, or images stored in the memory 50 or 68, or the data related to the displayed or stored images. The desired output may be in a tabular form, or images, that may be stored in the memory 50 or 68, for example. The desired output may be specified by the user. For example, the desired output may be specified by the user depending on the amount of adipose tissue to be reduced.

The feedback module 74 may compare the actual output and the desired output and inform/alert the system if required. In one example, the feedback module 74 may also use the therapy commands provided to the system 26 by the user to determine the acceptable levels of adipose tissue reduction and thermal treatment, and accordingly notify the system when such limits are exceeded or approaching. In one example, the feedback module 74 may alert the system by beeping, to caution the user, for example, if the determined limit of adipose tissue to be cavitated exceeds or is about to exceed a given parameter. In one embodiment, the feedback module 74 may have built-in intelligence that may alter the therapy parameters to amend the therapy being provided.

The feedback module 74 may take the data from the processing unit 48, displayed images (on the display 70), or memories 50 or 68 images, as the input and make a decision whether the data or images are acceptable. For example, the feedback module 74 may use the displayed images to determine whether the amount of adipose tissue cavitated in the region of interest 28 is sufficient to stop the therapy in the region of interest 28. The feedback module 74 may either provide feedback after completion of the therapy, or during the therapy. In one example, the feedback module 74 may verify whether the amount of the adipose tissue reduced from a given portion is acceptable by comparing the actual amount of the adipose tissue cavitated with the adipose tissue value calculated using the therapy parameters. If, for example, the depth of the adipose tissue ablated exceeds, or is about to exceed a determined value, the feedback module 74 may be configured to raise an alarm, such as a continuous beep, till the user acknowledges receiving the beep (for example by means of the user interface 64). The user may then review the information from the feedback module 74. In this manner, the feedback module 74 may avoid inadvertent errors that could otherwise occur due to user error.

The feedback module 74 may also use the therapy commands entered into the system 30 by the user to determine the acceptable levels of adipose tissue reduction and thermal treatment, and accordingly notify the system when such limits are exceeded or approaching.

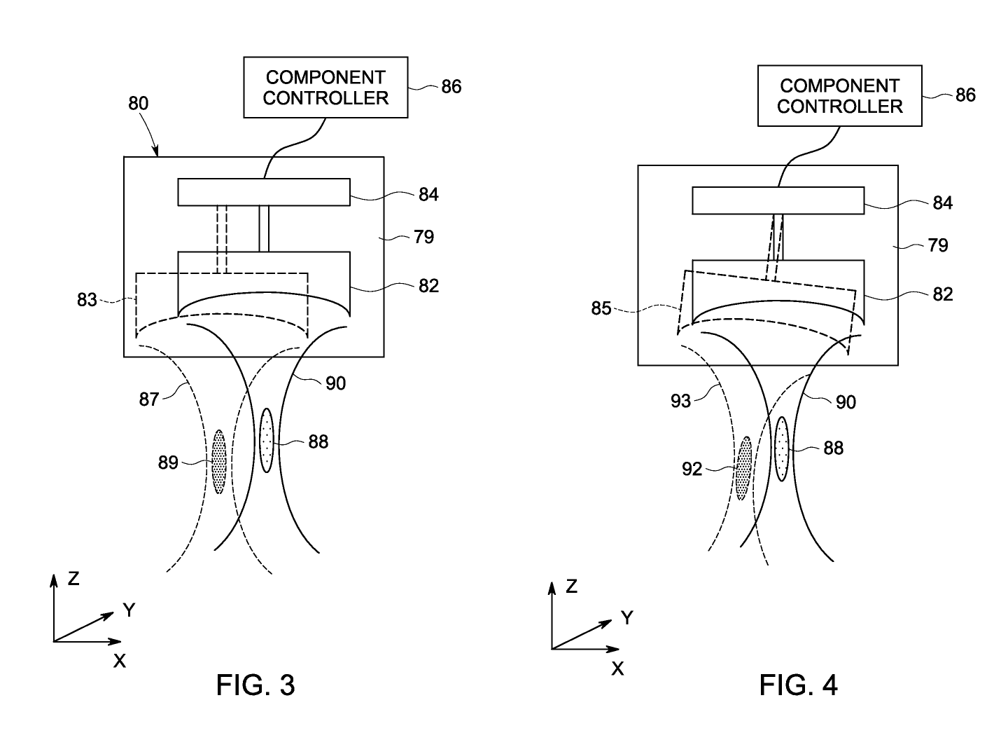

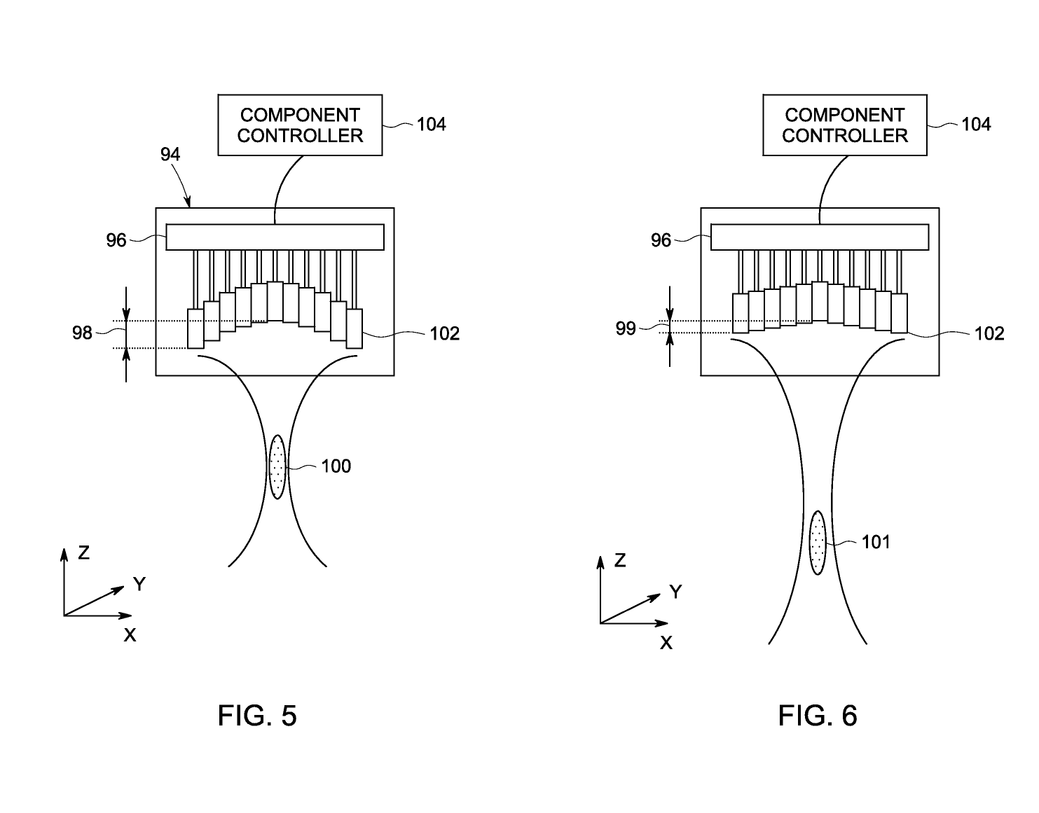

In certain embodiments, adjusting the position of the HIFU beam focus may be achieved by operatively coupling a beam enhancement component, such as an actuator assembly, to the ultrasound transducer assembly. The beam enhancement component may be directly or indirectly coupled to the transducer. In one example, the beam enhancement component may be physically disposed on the transducer as illustrated, for example, in the embodiments shown in FIGS. 3 and 4. Alternatively, the beam enhancement component may be acoustically coupled to the transducer or a structure that is directly coupled to the transducer. The beam enhancement component may be coupled to a shaft that is coupled to the transducer as illustrated, for example, in FIGS. 5 and 7-9. As illustrated in FIG. 6, instead of, or in addition to, being coupled to a shaft, the beam enhancement component may comprise a lens, such as an asymmetrical lens, that is in operative association with the transducer.

The beam enhancement component moves or rotates the beam, directly or indirectly, emitted from therapy transducer. The beam enhancement component may employ gear assemblies. In one embodiment, the beam enhancement component may be a stepper motor. A stepper motor is usually compact in size and is an electromagnetic device that converts electric pulses into discrete mechanical motion. The stepper motor employs a stator and a rotor. Fine control of the rotor position may be obtained by increasing number of detent positions on the rotor. The beam enhancement component may either employ an open loop command or close loop or feedback mode of command. The beam enhancement component may employ one or more stepper motors.

In certain embodiments, the beam enhancement component employing the motor, such as a stepper motor, may use micro stepping techniques to ensure smooth rotation and accurate position control of the transducer.

FIG. 3 illustrates an example of an ultrasound transducer assembly. The assembly 80 comprises a therapy transducer 82 that is operatively coupled to a beam enhancement component 84. Both the transducer 82 and the beam enhancement component 84 may be disposed in an acoustic coupling fluid 79. The beam enhancement component 82 operates based on the signals received from the component controller 86. The beam enhancement component 84 moves the transducer 82 in the x, y and z directions with respect to the assembly 80, thereby moving the position of the focus 88 of the ultrasound beam 90 in the x, y and z axes with respect to the assembly 80. A new location of the transducer 82 is represented by the reference numeral 83, the ultrasound beam 87 and the ultrasound beam focus 89 after a representative movement along the x and z directions. The acoustic coupling fluid 79 allows acoustic energy to be directed efficiently into the subject as the transducer 82 moves with respect to assembly 80.

Regions of interest with volumes larger than the transducer focus volume can be treated without moving the transducer assembly 80 by translating the transducer 82 laterally along or rotating about the x, y and z-axis. Translations and rotations can be used individually or in combination, to move and orient the transducer focus volume to generate a larger, more complicated, region of interest. For example, by translating the focus along one axis, a linear region of interest along that axis can be created. By moving the transducer a fixed amount along the x-axis, then rotating the transducer about the z-axis, a segment of a ring-like region of interest can be created. Any desired region of interest can be generated by suitable choices of translations and/or rotations.

The controller 90 provides commands or instructions or inputs to the driver member 84. For example, the controller 90 may switch the beam enhancement component 84 on and off. The controller 90 may be configured to receive user commands, and accordingly direct the driver member 84.

FIG. 4 illustrates an alternative embodiment in which the transducer 82 is moved to a different position represented by the reference numeral 85 by rotation of the transducer 82 about the x and y axis and by translation of the transducer 82 along the z axis. The resulting ultrasound beam 93 and focus 92 are displaced with respect to the original positions. The displacement of the focus 92 in the illustrated example is a result of rotating the transducer about the y-axis and translating the transducer along the z axis, thereby producing a new location 85 of the transducer 91 and the corresponding new position of the focus 92. In some applications, it may be easier and/or less expensive to use rotational actuators (for rotational movements) rather than translational actuators (for translational movements) to move the acoustic focus without moving the transducer assembly.

Referring now to FIG. 5, an ultrasound transducer assembly 94 comprises a beam enhancement component 96 and a therapy transducer 97. As illustrated, the transducer 97 comprises an array of transducer elements 102. The transducer elements 102 may be displaced or moved relative to each other to alter the position of the focal spot 100 of the ultrasound beam. In one example, the elements 102 may be displaced relative to each other along z-axis. The set of element displacements 98 along the z-direction moves the focal position of the ultrasound beam in the z-direction. The beam enhancement component 96 is controlled using a component controller 104.

In one embodiment, the array of the transducer elements 102 is in the form of concentric rings. The different rings may have different width. The width of the rings may be chosen to optimize the beamforming performance given a desired number of rings and a desired range of focus depths. In this embodiment, one or more of these concentric rings can be displaced relative to each other under the control of component controller 104. By adjusting the relative positions of the rings so that inner surface approximates a sphere of a particular radius, the depth of the focal region can be adjusted.

FIG. 6, an ultrasound transducer assembly comprises a beam enhancement component 96 and a therapy transducer 95. As illustrated, the transducer 95 comprises an array of transducer elements 103. The transducer elements 102 may be displaced or moved relative to each other to alter the position of the focal spot 101 of the ultrasound beam.

FIG. 5 illustrates the case in which the radius of the inner surface is relatively small, as represented by the reference numeral 98, so that the focus 100 lies relatively near the transducer housing. FIG. 6 illustrates the case in which the radius is larger, as represented by the reference numeral 99, so that the focus 101 lies farther from the transducer housing. Unlike the situation for phased arrays, the same electrical drive signal can be used for each ring, since the required focusing time delays are created geometrically. FIGS. 5 and 6 illustrate the case in which the transducer is divided into annular ring elements. The same principles apply to the case in which the transducer is divided into a one-dimensional array of elements, which allows focal depth control and steering control in one axis, and to the case of a two-dimensional array of elements, which allows focal depth control and steering control in two axes.

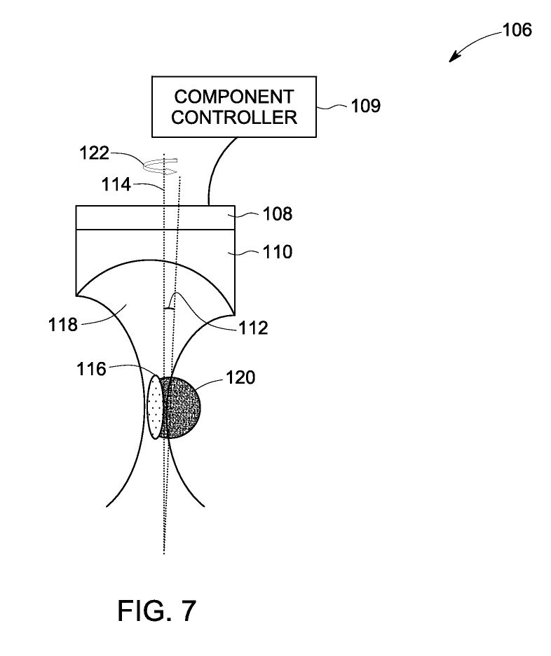

FIG. 7 illustrates an ultrasound transducer assembly 106 having a transducer 110 and a beam enhancement component 108. The transducer 110 may be a single element transducer. The transducer 110 is inclined at an angle 112 with respect to a central axis or axis of rotation 114. The inclination of the transducer 110 about the central axis 114 moves the focal spot 116 of the beam 118 from transducer 110, off-axis with respect to the central axis 114. The angle 112 of inclination of the transducer 110 may be in a range from about 0 degrees to about 30 degrees. The angle of inclination depends on the focal spot size, the focal depth, and the amount of overlap in the focal spot desired as the transducer rotates.

As represented by curved arrow 122, the transducer 110 may be rotated about the central axis 114. A component controller 109 may be used to control the motion of the beam enhancement component 108 and in turn the motion of the transducer 110. A portion of the three-dimensional region covered by the focal spot 116 due to rotation of the transducer 110 about the central axis 114 is represented by the reference numeral 120. The inclination of the transducer 110 relative to the central axis 114 enables a larger coverage area per rotation of the transducer 110. The larger coverage area per rotation of the transducer 110 reduces the overall time for therapy without increasing the probe size. Also, since a physical movement of the probe is not required to cover the larger area, the time, otherwise required for adjusting the probe to the new location to focus the beam to a desired point in the location, is saved.

In one embodiment, the angle of inclination 112 of the transducer 110 may be maintained constant during the operation. For example, the angle of inclination 112 may be maintained constant for a circular region of interest. In another embodiment, the angle of inclination 112 of the transducer 110 may be varied at least during a portion of the operation of the transducer 110. For example, for a region of interest having ellipsoidal shape, the angle of inclination 112 may be varied periodically during each rotation of the transducer. In one example, initially the transducer 110 may not be inclined with respect to the central axis 114, that is, the initial angle of inclination may be at zero degrees. However, during delivery of the therapy or during imaging, the angle of inclination may be changed to a different value. The angle of inclination may be changed gradually in small steps. For example, the angle of inclination may be changed by a small value after each rotation. Alternatively, the angle of inclination may be directly changed from one desired value directly to another desired value. In one embodiment, the rotation of the transducer 110 inclined at an angle may be combined with a lateral movement of the transducer. For example, the transducer 110 may be laterally shifted along one or more of x or y-axis before making the next rotation of the transducer 110 to cover more than one circular or ellipsoidal region of interest.

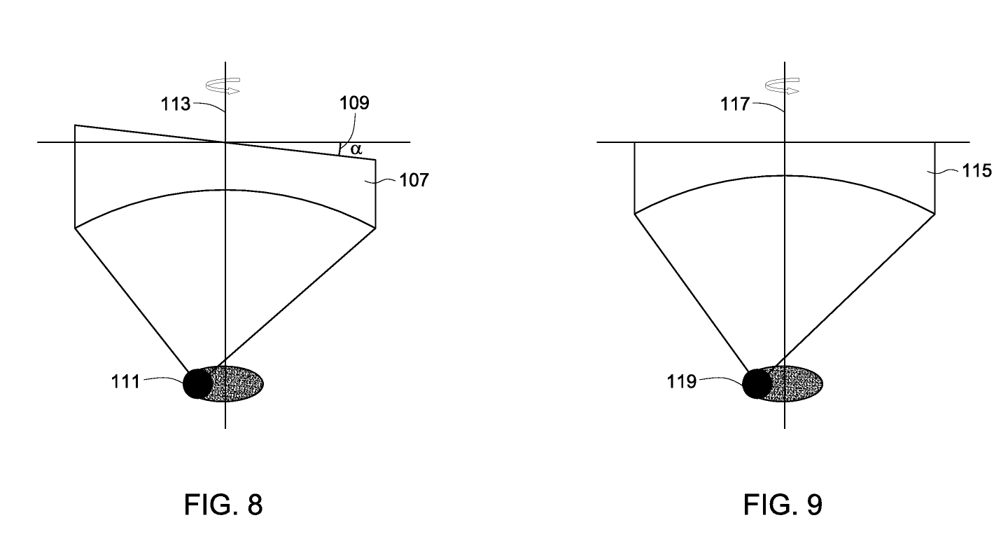

An alternative to using a transducer with an incline is to begin with a transducer that is asymmetric in such a way that the geometric focus is off-axis (relative to the axis of rotation). FIG. 8 illustrates a physically symmetric transducer 107 with an inclination 109 to make the focus 111 of the transducer 107 off-axis with respect to the axis of rotation 113. FIG. 9 illustrates another transducer 115 with off-axis focus, the shape of the transducer 115 is asymmetric about the axis of rotation 117. The asymmetric shape of the transducer 115 makes the focus 119 of the transducer off-axis with respect to the axis of rotation 117. The off-axis focus for the transducers of FIGS. 8-9 enable greater coverage area by the focus thereby covering larger treatment region when the transducer is rotated about a central axis or axis of rotation.

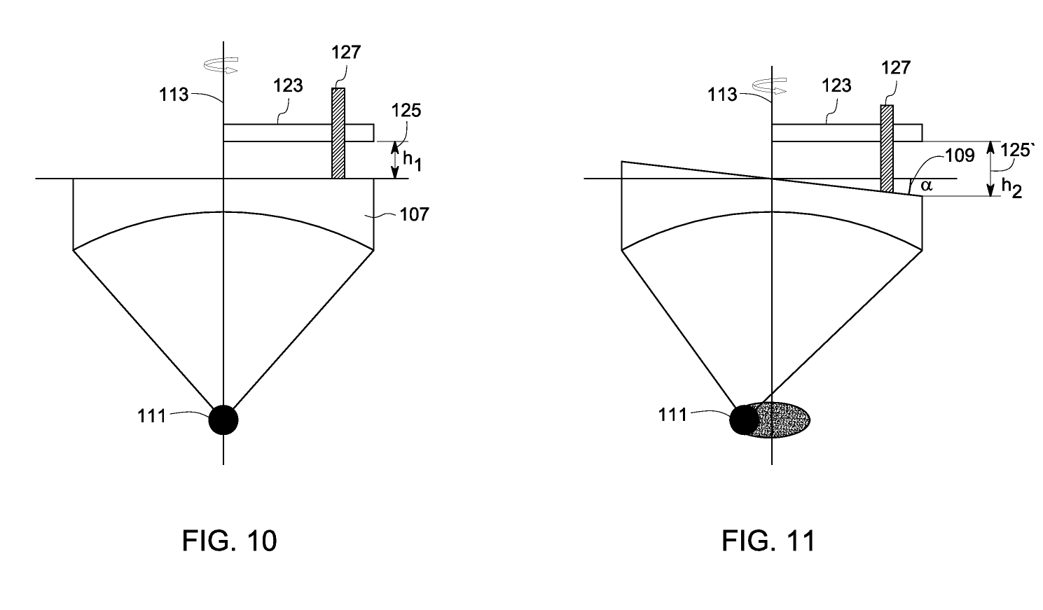

FIGS. 10-11 illustrate a beam enhancement component for the transducer assembly of FIG. 8. The beam enhancement component is composed of two actuators: a first actuator 123 for causing the inclination of the transducer 107, and a second actuator (not shown) for rotating the inclined transducer 107. The first actuator 123, which is a linear actuator, controls the height, h 125, of a contact pin 127 that is in contact with the transducer 107. Change in the height 125 of the pin 127 results in change in the angle of inclination 109 of the transducer. However, in the case of FIG. 9, the focus is already off-axis due to the asymmetric shape of the transducer, hence, only single actuator may be required to rotate the asymmetric transducer.

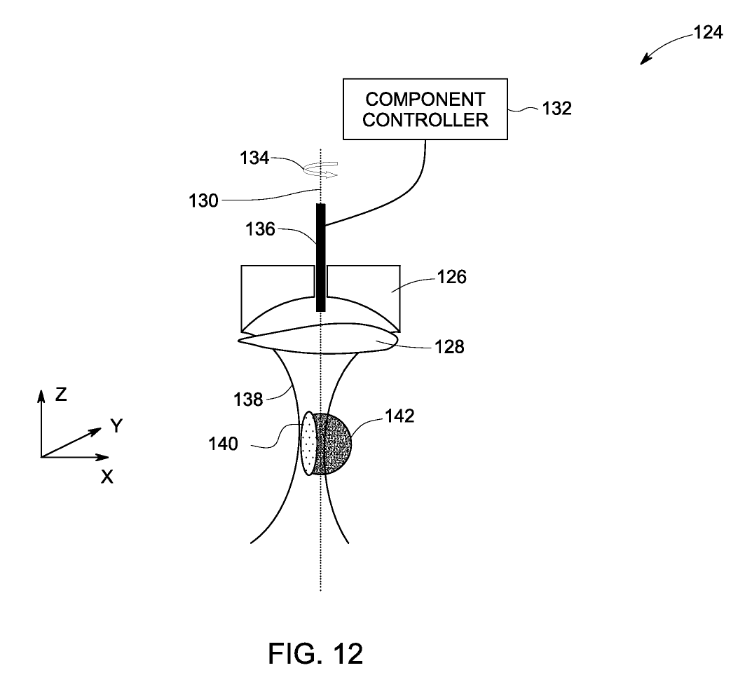

FIG. 12 illustrates an ultrasound transducer assembly 124 that employs a transducer 126 and a beam enhancement component, which is an asymmetric acoustic lens 128. The transducer 124 may be stationary about the central axis 130. Further, an asymmetric acoustic lens 128 is in operative association with the transducer 126. The asymmetric lens 128 is dimensioned to exhibit non-uniform thickness for refracting the ultrasonic beam in a predetermined manner. In one example, the asymmetric lens 128 may be dimensioned to change the focal depth of the ultrasonic beam 138.

The beam enhancement component, that is the asymmetric lens 128, rotates about the central axis 130 of the transducer and the lens 128. The transducer 126 and the lens 128 may or may not have overlapping central axis. Curved arrow 134 represents the rotational movement of the lens 128. A shaft 136 is used to hold the asymmetric lens 128 in place and enable the rotation of the lens 128. The rotation of the lens 128 is controlled by the component controller 132. For the ease of assembly, the shaft 136 may coincide with the central axis 130 of the transducer 126.

The asymmetric lens 128 transmits the ultrasonic beam and focuses the beam 138 at a focus spot 140. Rotation of the lens 128 causes a change of the ultrasonic beam path in the lateral direction or the depth dimension or both. As the asymmetric lens 128, in the form of a scanning disk (due to rotation), undergoes rotational motion in a plane normal to the path of the ultrasonic beam 138, for example at a uniform speed, the ultrasonic beam 138 encounters a steadily changing lens thickness, thus causing the beam to exhibit a repetitive pattern of varying beam path and/or focal depth. As the disk undergoes motion the effect of an infinite sequence of lenses each of different contour intercepting the ultrasonic beam in a plane substantially normal to the ultrasonic beam 138 is manifest. In the illustrated embodiment, when the beam 138 passes through the asymmetric lens 128, the focus of the beam shifts off axis from the central axis 130. During the rotation of the asymmetric lens 128, the focal spot 140 also makes a corresponding rotation, because of the focal spot 140 being off-axis a larger area is covered by the beam 138. Reference numeral 142 represents a portion of the three-dimensional area scanned by the focal spot 140 during rotation of the lens 128. The scanned area may be in the form of an ellipsoid or circle, or any other geometric shape.

Although not illustrated, in addition to exhibiting varying thickness, the lens 128 may have one or more contoured grooves. By proper contouring of the surface of the asymmetric lens 128, the ultrasonic beam 138 may be refracted to scan the region of interest.

In certain embodiments, the transducer 126 may be laterally shifted along one or more of x, y or z-axis before making the next rotation of the lens. The lateral shifting requires a larger probe housing and may not be desirable for smaller probe housings. Alternatively, the transducer 126 may be laterally shifted along one or more of x, y or z-axis after a determined number of rotations of the lens 128. The lateral and the rotational movements may be controlled by component controller 132. Although not illustrated, the component controller 132 may also be coupled to the transducer 126 to enable any lateral movements of the transducer 126. A mechanical drive may be employed by the lens 128 or the transducer 126 to enable the motion of the lens 128 or the transducer 126, where the mechanical drive may receive control signals from the component controller 132.

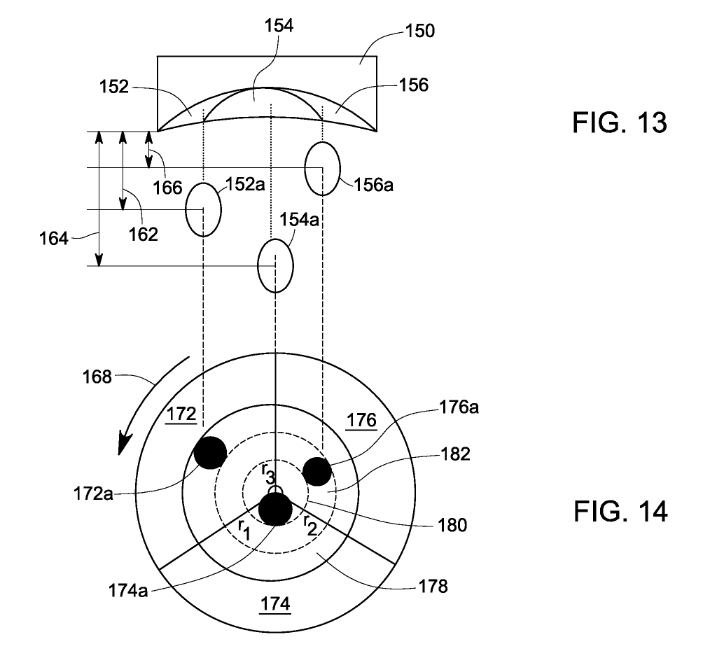

FIG. 13 illustrates a sectioned transducer element 150. The transducer 150 may have a single element or an array of elements. The transducer may be divided into, for example, two or more portions or sectors, with each portion having a different focal location. In the illustrated embodiment, the transducer is shown to be sectioned into three portions 152, 154 and 156 with each section having its own focus indicated by 152a, 154a, and 156a, respectively. The foci 152a, 154a, and 156a have focal depths indicated by 162, 164 and 166, respectively. The different focal depths 162, 164 and 166 may be employed for different applications requiring different focal depths. Depending on the depth of the region of interest a specific part of the portioned/sectioned transducer may be activated followed by translation or rotation. Sectors that are not required for the particular treatment may even be switched off. Although not illustrated, the transducer 150 may be coupled to a beam enhancement component and a component controller to enable rotation of the transducer. The transducer 150 may or may not be inclined at an angle relative to a central axis of the transducer 150.

As illustrated in FIG. 14, which is a top-down view of FIG. 13, the transducer 150 is rotated or oscillated about an axis as illustrated by the arrow 168. In the illustrated embodiment, the transducer 150 is oscillated about the central axis. The three different sectors 172, 174 and 176 correspond to sectors 152, 154, and 156 respectively, of FIG. 13. Sectors 172, 174 and 176 each have foci indicated by 172a, 174a and 176a. Foci 172a, 174a and 176a correspond to foci 152a, 154a and 156a, respectively, of FIG. 13. A complete 360 degrees rotation of transducer 150 causes foci 172a, 174a and 176a to sweep out the annuli indicated by 178, 182 and 180 respectively. The annular areas 178, 180 and 182 may or may not be overlapping. The illustrated embodiment of FIGS. 13-14 may enable continuous coverage area, and therefore, may be useful for treatments involving ablation of diseased tissues, such as cancerous tissues. The rotation speed, or RPM of rotation may be decided by time required for treating a particular region of interest. For example, the rpm may depend upon the time required to ablate the adipose tissue. The foci 172a, 174a, 176a are shown in FIG. 14 as each having a different radius from the central axis. Alternatively, the foci may have the same radius from the central axis (not shown), and vary only in the focal depth.

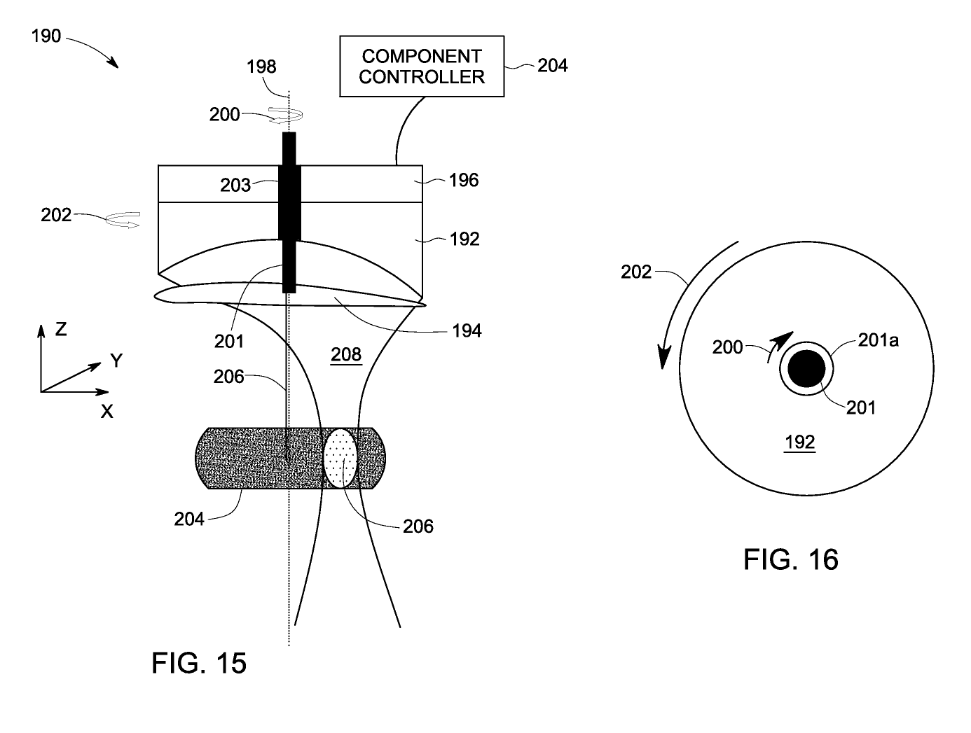

FIG. 15 illustrates an ultrasound transducer assembly. The ultrasound transducer assembly 190 comprises a transducer 192. The assembly 190 further comprises beam enhancement components: an acoustic asymmetric lens 194 and a mechanical drive member 196 in operative associations with the transducer 192. In addition, the transducer 192 may be rotated about the central axis 198 as represented by the arrow 202 by employing a shaft 203. The lens is coupled to a shaft 201 that may also rotate about central axis 198. In order to steer the ultrasound beam off axis, the transducer 192 may be a symmetrical transducer inclined at an angle with respect to the central axis 198 as in FIG. 8, or may be an asymmetrical transducer with off-axis focus as in FIG. 9. The transducer 192 and the asymmetric lens 194 may rotate or oscillate independent of each other, thereby enabling increased control of the placement of the focal spot. Depending on the size and shape of the region of interest that needs to be covered by the focal spot 206, either one or both the transducer 192 and the lens 194 may rotate and/or oscillate. In addition, the transducer 192 may be laterally moved along one or more axis. For example, if the region to be covered is small, only one of the transducer 192 or the lens 194 may need to be rotated. The motion of the beam enhancement components 194 and 196 may be controlled by the component controller 204.

The combined effect of the inclination or asymmetry of the transducer 192 and the use of asymmetric lens 194 causes the focal spot 206 to be off axis by a greater degree, when compared to the focal spots of other configurations. The greater degree of offset of the focal spot 206 enables larger coverage area during rotation of one, or both, of the transducer 192 and the lens 194. In one embodiment, the transducer 192 and the lens 194 may be rotated in the same direction. As illustrated in FIG. 16, in another embodiment, the transducer 192 and the lens 194 may be rotated in opposite directions 200 and 202.

When the direction of beam steering due to the asymmetry or inclination of transducer 192 is in the same direction of beam steering due to the asymmetric lens 194, the net effect is that the beam is steered off-axis to a maximum extent. When the direction of beam steering due to the asymmetry or inclination of transducer 192 is in the opposite direction of beam steering due to the asymmetric lens 194, the net effect may be that the beam remains on-axis. By varying the rotation of the transducer 192 and the lens 194 relative to one another, a continuous range of beam steering between 0 degrees and the maximum off-axis amount is possible. Thus, a larger treatment region may be achieved from a single physical transducer location.

Reference numeral 204 represents a side view of an example of a region of interest being covered by rotation of the transducer 192 and the lens 194. The speed of rotation of the transducer 192 and the lens 194 may be same or different and may depend upon the desired radius and required time for a given coverage area. In addition, the lens 194 may be moved along the z-axis to adjust the focal depth 204 of the beam 208.

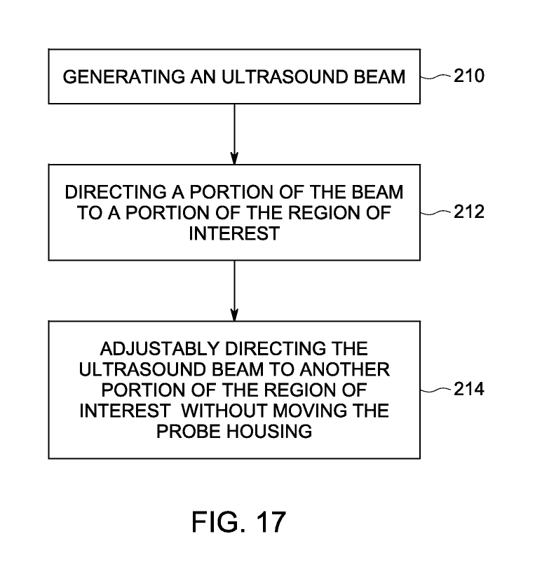

FIG. 17 is a block diagram illustrating various steps in an example of a method for treating and/or imaging a region of interest using ultrasound. The method of this example comprises non-invasively treating the region of interest. The method enables treating and/or imaging one or more regions of interest with minimal or no displacement of the probe housing, which would otherwise be required in conventional methods and ultrasound systems. For example, the number of probe movements required using a conventional ultrasound system and method may be more than the number of movements required by the present method and system. Since each probe movement is followed by adjustment time, where the probe position is adjusted to be able to focus the beam at the desired location, lesser number of probe movements adds up to lesser overall adjustment time while increasing the accuracy of the system. The beam adjustment, such as beam steering and focusing, of the ultrasound beam to cover larger regions of interest reduces the need for physical probe housing displacement. The method provides enhanced control of focal spot positioning in the region of interest, and movement of the focal spot from one region of interest to another, or from one portion of a particular region of interest to another portion.

At block 210, a probe housing is disposed on a surface corresponding to the region of interest in the subject, wherein the probe housing comprises a transducer. The transducer generates an ultrasound beam having determined beam parameters. At block 212, acoustic energy is directed to at least a portion of the region of interest using the probe.

At block 214, the ultrasound beam is adjustably directed to another portion of the region of interest without moving the probe housing physically on the patient. In one embodiment, the step of adjustably directing the ultrasound beam comprises changing one or beam parameters including a shape, a size or a direction of the ultrasound beam relative to the region of interest. The beam characteristics may be altered by activating the beam enhancement component and/or the transducer. The transducer position or orientation, or both, can be varied to alter beam parameters. For example, the transducer can move along one or more of x-axis, y-axis, or z-axis for adjusting the beam with respect to one or more beam parameters. In addition, or alternately, the transducer may also rotate along one or more of the axis. In certain embodiments, a beam enhancement component may be coupled to the transducer to enable the transducer movements in a desired fashion. The beam adjustment may be performed dynamically. In other words, the beam steering may be performed while continuing the therapy.

The beam adjustment may comprise inclining the transducer along a transducer axis, rotating the transducer along the transducer axis, rotating a lens relative to the transducer, wherein the lens is disposed between the transducer and the region of interest, and/or steering the direction of the acoustic energy by moving the transducer element about an axis. The movement of the transducer element or the array of elements may be controlled by a computer using algorithms to adjustably direct the ultrasound beam to desired locations without physically moving the probe housing relative to the subject.

In certain embodiments, the ultrasound transducer assembly may scan through different scan planes. The ultrasound transducer may move along the x-axis, y-axis, z-axis, or combinations thereof. In one embodiment, the ultrasound transducer rotates along z-axis. The ultrasound transducer, in this example, comprises a lens acoustically coupled to the transducer. An input face of the lens is positioned to receive the ultrasound energy, and focus it to a desired location. In one embodiment, an input face of the lens may be positioned at an angle with respect to the transducer axis to receive the ultrasonic beam and to pass the ultrasonic energy to pass through the lens. The lens may be used to focus and/or steer the ultrasound energy for selectively controlling the focusing and/or steering of the ultrasonic energy within a region of interest in an object.

In one embodiment, the lens is an asymmetric lens. The lens rotates or oscillates about a center axis. The lens may also move along the center axis. In one embodiment, the lens and the transducer rotate or oscillate about the same axis. The lens and the transducer rotate may rotate in the same or opposite directions. The transducer may be inclined at an angle with respect to the central axis.

Various embodiments of the methods and systems of the invention combine the simplicity and cost-effectiveness of a single channel system with the sophistication of a phased array system in providing beam adjustment. Various systems and methods of the invention provide economical solutions for HIFU based therapies, and are capable of delivering therapy in a time efficient.

While only certain features of the invention have been illustrated and described herein, many modifications and changes will occur to those skilled in the art. It is, therefore, to be understood that the appended claims are intended to cover all such modifications and changes as fall within the scope of the invention.

1. An ultrasound transducer assembly for adjustably directing a beam of ultrasound energy to a region of interest, comprising:
a therapy transducer;
a beam enhancement component that adjusts one or more parameters of the ultrasound beam, wherein the beam enhancement component is in operative association with the therapy transducer; and
a component controller operatively coupled to the beam enhancement component for controlling the beam enhancement component.

2. The ultrasound transducer assembly of claim 1, wherein the beam enhancement component comprises a mechanical drive member that moves the therapy transducer to adjust one or more of the parameters of the ultrasound beam.

3. The ultrasound transducer assembly of claim 1, wherein the beam enhancement component is disposed on the therapy transducer.

4. The ultrasound transducer assembly of claim 1, wherein the therapy transducer and/or the beam enhancement component are disposed in a coupling fluid.

5. The ultrasound transducer assembly of claim 1, wherein the therapy transducer comprises a transducer element.

6. The ultrasound transducer assembly of claim 1, wherein the therapy transducer comprises an array of transducer elements.

7. The ultrasound transducer assembly of claim 1, wherein the therapy transducer is inclined at an angle relative to a central axis.

8. The ultrasound transducer assembly of claim 1, wherein the therapy transducer is asymmetric such that a focus of the transducer is away from a central axis.

9. The ultrasound transducer assembly of claim 1, wherein the therapy transducer rotates or oscillates about a central axis.

10. The ultrasound transducer assembly of claim 1, wherein the therapy transducer moves about one or more of an x-axis, y-axis, z-axis, or combinations thereof.

11. The ultrasound transducer assembly of claim 1, wherein the therapy transducer is divided in two or more sections wherein at least one of the sections has a focal depth that is different from the focal depth of at least one of the other sections.

12. The ultrasound transducer assembly of claim 1, wherein the therapy transducer is divided in two or more sections wherein at least one of the sections has a focus with a radius from the central axis (focal radius) that is different from the focal radius of at least one of the other sections.

13. The ultrasound transducer assembly of claim 1, further comprising a compliant member disposed between the therapy transducer and the beam enhancement component.

14. The ultrasound transducer assembly of claim 1, comprising a single channel.

15. The ultrasound transducer assembly of claim 1, wherein the beam enhancement component comprises an asymmetric lens at least acoustically coupled to the therapy transducer.

16. The ultrasound transducer assembly of claim 15, wherein the lens moves about one or more axis to adjust one or more of the parameters of the ultrasound beam.

17. The ultrasound transducer assembly of claim 15, wherein the lens rotates or oscillates about an axis.

18. The ultrasound transducer assembly of claim 15, wherein both the lens and the therapy transducer rotate or oscillate about the same axis.

19. The ultrasound transducer assembly of claim 18, wherein the lens and the therapy transducer rotate in opposite directions.

20. The ultrasound transducer assembly of claim 1, wherein the ultrasound transducer assembly is disposed in a probe housing.

21. An ultrasound transducer system, comprising:
a probe housing;
an ultrasound transducer assembly at least partially disposed in the probe housing, comprising:
a therapy transducer;
a beam enhancement component that adjusts one or more parameters of the ultrasound beam, wherein the beam enhancement component is in operative association with the therapy transducer;
a component controller operatively coupled to the beam enhancement component for controlling the beam enhancement component; and
a therapy module that provides one or more therapy parameters to the ultrasound transducer assembly.

22. A method for non-invasively providing therapy to a region of interest using an ultrasound transducer associated with a probe housing, comprising:
generating an ultrasound beam;
directing at least a portion of the beam to a portion of the region of interest; and
adjustably directing the same portion, or a different portion, of the ultrasound beam to another portion of the region of interest without moving the probe housing.

23. The method of claim 22, wherein adjustably directing the ultrasound beam comprises changing a shape, a size or a direction of the ultrasound beam relative to the region of interest.

24. The method of claim 22, comprising moving a position or an orientation, or both, of the transducer within the probe housing

25. The method of claim 22, comprising delivering therapy to two or more regions of interest wherein some of the regions of interest comprise overlapping areas.

26. The method of claim 25, wherein the regions of interest are concentrically disposed.

27. The method of claim 22, wherein the step of adjustably directing the ultrasound beam comprises inclining the therapy transducer along a transducer axis.

28. The method of claim 22, wherein the step of adjustably directing the ultrasound beam comprises rotating the therapy transducer about a transducer axis.

29. The method of claim 22, wherein the step of adjustably directing the ultrasound beam comprises rotating an asymmetric acoustic lens relative to the therapy transducer, wherein the lens is disposed between the therapy transducer and the region of interest.

30. The method of claim 29, wherein the step of adjustably directing the ultrasound beam comprises rotating an asymmetric therapy transducer.

31. The method of claim 22, wherein the step of adjustably directing the ultrasound beam comprises moving at least a portion of the therapy transducer, or the beam enhancement component, or both, about an axis.

32. The method of claim 22, wherein the step of adjustably directing the ultrasound beam comprises activating a determined portion of a sectioned transducer depending on the depth of the region of interest.

33. The method of claim 22, wherein the step of adjustably directing the ultrasound beam comprises, moving one or more of a plurality of transducer elements relative to each other.
